(19) United States
(12) Patent Application Publication
(10) Pub. No.: US 2024/0110138 A1
(43) Pub. Date: Apr. 4, 2024

(54) MICRO ELECTRODE ARRAY DEVICE AND METHOD WITH TEMPERATURE CONTROL

(71) Applicant: SAMSUNG ELECTRONICS CO., LTD., Suwon-si (KR)

(72) Inventors: SEONG JOONG KIM, Suwon-si (KR); CHISUNG BAE, Suwon-si (KR)

(73) Assignee: SAMSUNG ELECTRONICS CO., LTD., Suwon-si (KR)

(21) Appl. No.: 18/185,557

(22) Filed: Mar. 17, 2023

(30) Foreign Application Priority Data
Oct. 4, 2022 (KR) 10-2022-0126323

(51) Int. Cl. C12M 1/12; C12M 1/34; C12M 1/36; C12M 3/00

(52) U.S. Cl. CPC C12M 23/04; C12M 3/00; C12M 41/12; C12M 41/48

(57) ABSTRACT

A micro electrode array (MEA) platform including a cell culture container configured to accommodate a cell culture medium to culture neurons, an MEA including an electrode configured to sense the neurons, and a temperature control device configured to control a transfer of heat generated by a heating source through a heating wire, based on whether a temperature of the cell culture medium or the electrode is equal to a reference temperature for the neurons.

### CROSS-REFERENCE TO RELATED APPLICATIONS

This application claims the benefit under 35 USC § 119(a) of Korean Patent Application No. 10-2022-0126323, filed on Oct. 4, 2022, in the Korean Intellectual Property Office, the entire disclosure of which is incorporated herein by reference for all purposes.

### BACKGROUND

### 1. Field

The following description relates to a micro electrode array device and method with temperature control.

### 2. Description of Related Art

Various methods may be applied to analyze a person's vital signs. For example, signals of neurons that are measured by electrodes may be used to analyze brain waves. In addition, a certain amount of current passes through neurons of the brain nerve. These currents may be observed to study intracellular signals and to understand how the brain transmits signals. Temperature may correspond to one of the vital signs or physiological parameters and the temperature of neurons may play an important role in life activities. Temperature may, for example, affect various processes inside a cell, ranging from gene expression to protein interactions. Therefore, properly maintaining a cell temperature may help brain neurons actively generate signals.

The above description has been possessed or acquired by the inventor(s) in the course of conceiving the present disclosure and is not publicly known before the present application is filed.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In one general aspect, there is provided a micro electrode array (MEA) platform including a cell culture container configured to accommodate a cell culture medium to culture neurons, an MEA including an electrode configured to sense the neurons, and a temperature control device configured to control a transfer of heat generated by a heating source through a heating wire, based on whether a temperature of the cell culture medium or the electrode is equal to a reference temperature for the neurons.

The temperature control device may include a temperature sensor configured to detect the temperature of the cell culture medium and the electrode, a heating module configured to heat at least one of the electrode or the cell culture medium by the heat generated by the heating source, and a controller configured to control the heating module based on whether the temperature is equal to the reference temperature, and to transfer the heat to at least one of the electrode or the cell culture container through the heating wire.

The temperature control device further may include a cooling module configured to cool at least one of the electrode or the cell culture medium, and the controller may be configured to drive the cooling module to cool a temperature of the electrode and a temperature of the cell culture medium, in response to the temperature being greater than the reference temperature, and drive the heating module to increase the temperature of the electrode and the temperature of the cell culture medium, in response to the temperature being lesser than the reference temperature.

The MEA platform may be packaged in a chip form on a printed circuit board (PCB) using one or more of an interposer and a bonding wire, and the cell culture container may be disposed on an upper surface of the chip.

The heating source may include an on-chip variable resistor, and the controller may be configured to determine a difference between the temperature and the reference temperature and to set a resistance value of the variable resistor according to the difference.

The heating module may include a heating loop made of a metal wire, wherein the heating loop may cross boundaries of at least one of an interposer, a printed circuit board (PCB), or a bonding wire in an upper portion of a metal layer positioned adjacent to the electrode.

The heating loop structure may be shaped to surround a bottom surface of the cell culture container.

The MEA platform may include an expanded metal heating wire extending from the heating loop to be disposed along a perimeter of the cell culture container, wherein the expanded metal heating wire may be configured to transfer heat generated through the variable resistor to surroundings of the cell culture container.

A cooling module including a cooling fan may be disposed outside the cell culture container, wherein the controller may be configured to drive the cooling fan to lower the temperature of the cell culture medium, in response to the temperature of the cell culture medium being greater than the reference temperature, and generate heat through the variable resistor to increase the temperature of the cell culture medium, in response to the temperature of the cell culture medium being lesser than the reference temperature.

The heating module may be configured to transfer the heat generated by the heating source, by heat transfer lines, and wherein each of the heat transfer lines may be disposed to cross boundaries of at least one of an interposer, a printed circuit board (PCB), or a bonding wire in an upper portion of a metal layer positioned adjacent to the electrode.

The temperature control device may include a heat dissipation plate disposed adjacent to a high current line where the heat is generated more than a threshold amount, and the heat dissipation plate may be disposed adjacent to a heat dissipation layer in a lower portion of a metal layer of a printed circuit board (PCB) on which the MEA platform is packaged.

The temperature control device may include a cooling fan disposed outside the cell culture container, and the controller may be configured to cool the cell culture medium or the heat dissipation plate by activating the cooling fan.

The heat dissipation plate may be at least one of a vertical heat dissipation plate configured in a form of surrounding a chip package and a horizontal heat plate configured on a plane, and may be connected to at least one of a chip in which the MEA platform may be packaged, an interposer, a bonding wire, and a PCB.

In another general aspect, there is provided a method of operating a temperature control device of a micro electrode array (MEA) platform, the method including detecting temperature of at least one of a cell culture medium culturing neurons or an electrode of an MEA configured to sense the neurons, determining whether the detected temperature may be equal to a reference temperature for the neurons, and controlling a transfer of heat generated by a heating source through a heating wire, in response to the detected temperature not being equal to the reference temperature.

The controlling of the transfer of the heat may include driving a cooling module to cool the electrode and the cell culture medium, in response to the detected temperature being greater than the reference temperature, and driving a heating module including the heating source to heat the electrode and the cell culture medium, in response to the detected temperature being lesser than the reference temperature.

The heating source may include a variable resistor, and the controlling of the transfer of the heat may include determining a difference between the detected temperature and the reference temperature, determining a heating intensity according to the difference, and setting a resistance value of the variable resistor according to the heating intensity.

The transfer of the heat may include determining a difference between the detected temperature and the reference temperature, determining a cooling intensity according to the difference, and cooling at least one of the electrode or the cell culture medium according to the cooling intensity.

In another general aspect, there is provided a device including a chip installed on a surface of a printed circuit board (PCB), the chip including a micro electrode array (MEA) configured to sense signals from biological matter, a temperature sensor configured to measure a temperature of the biological matter, a heating module configured to heat the biological matter based on the temperature, and a controller configured to control the heating module, a cell culture container disposed on an upper surface of the chip, the cell culture container being configured to store the biological matter in a cell culture medium, and an interface configured to output the signals.

The heating module may include a variable resistor disposed under the MEA, and the controller may be configured to vary a resistance of the variable resistor according to a difference of the temperature and a reference temperature for the biological matter.

The device may include a cooling module configured to cool the biological matter based on the temperature, and the controller may be configured to selectively drive one of the heating module and the cooling module based on a comparison of the temperature with a reference temperature for the biological matter.

Other features and aspects will be apparent from the following detailed description, the drawings, and the claims.

### BRIEF DESCRIPTION OF THE DRAWINGS

FIG. 1A illustrates an example of a micro electrode array (MEA) platform.

FIG. 1B illustrates an example of a temperature control device included in the MEA platform.

FIG. 2A to 2C illustrate an example of a chip in which an MEA platform is packaged.

FIG. 3 illustrates an example of a chip in which an MEA is packaged and a cell culture container.

FIGS. 4A to 4C illustrate examples of an arrangement of a heating wire in a heating module.

FIGS. 5A-5B illustrate examples of an arrangement of a heating wire in a heating module.

FIGS. 6A-6C illustrate examples of an arrangement of a heating wire in a heating module.

FIG. 7 illustrates an example of an arrangement of a heating module and a cooling module in an MEA platform.

FIG. 8 illustrates an example of an MEA platform including a heat dissipation plate.

FIG. 9 illustrates an example of a cooling module including a heat dissipation plate and a cooling fan.

FIG. 10 illustrates an example arrangement of metal layers of a chip, in which an MEA platform is packaged, and a printed circuit board (PCB).

FIGS. 11A and 11B illustrate examples of heating wire arrangements of a heating module in an MEA platform.

FIGS. 12A and 12B illustrate examples of arrangements of a heating module and a cooling module in an MEA platform.

FIGS. 13A and 13B illustrate examples of arrangements of a heating wire of a heating module, and a cooling fan and heat dissipation plates of a cooling module in an MEA platform.

FIG. 14 illustrates an example of a method of operating a temperature control device.

FIGS. 15 and 16 illustrate examples of a method of operating a temperature control device.

Throughout the drawings and the detailed description, unless otherwise described or provided, the same or like drawing reference numerals will be understood to refer to the same or like elements, features, and structures. The drawings may not be to scale, and the relative size, proportions, and depiction of elements in the drawings may be exaggerated for clarity, illustration, and convenience.

### DETAILED DESCRIPTION

The following detailed description is provided to assist the reader in gaining a comprehensive understanding of the methods, apparatuses, and/or systems described herein. However, various changes, modifications, and equivalents of the methods, apparatuses, and/or systems described herein will be apparent after an understanding of the disclosure of this application. For example, the sequences of operations described herein are merely examples, and are not limited to those set forth herein, but may be changed as will be apparent after an understanding of the disclosure of this application, with the exception of operations necessarily occurring in a certain order. Also, descriptions of features that are known after an understanding of the disclosure of this application may be omitted for increased clarity and conciseness.

The features described herein may be embodied in different forms and are not to be construed as being limited to the examples described herein. Rather, the examples described herein have been provided merely to illustrate some of the many possible ways of implementing the methods, apparatuses, and/or systems described herein that will be apparent after an understanding of the disclosure of this application.

Although terms such as “first,” “second,” and “third”, or A, B, (a), (b), and the like may be used herein to describe various members, components, regions, layers, portions, or sections, these members, components, regions, layers, portions, or sections are not to be limited by these terms. Each of these terminologies is not used to define an essence, order, or sequence of corresponding members, components, regions, layers, portions, or sections, for example, but used merely to distinguish the corresponding members, components, regions, layers, portions, or sections from other members, components, regions, layers, portions, or sections. Thus, a first member, component, region, layer, portions, or section referred to in the examples described herein may also be referred to as a second member, component, region, layer, portions, or section without departing from the teachings of the examples.

Throughout the specification, when a component or element is described as being “connected to,” “coupled to,” or “joined to” another component or element, it may be directly “connected to,” “coupled to,” or “joined to” the other component or element, or there may reasonably be one or more other components or elements intervening therebetween. When a component or element is described as being “directly connected to,” “directly coupled to,” or “directly joined to” another component or element, there can be no other elements intervening therebetween. Likewise, expressions, for example, “between” and “immediately between” and “adjacent to” and “immediately adjacent to” may also be construed as described in the foregoing. As used herein, the term “and/or” includes any and all combinations of one or more of the associated listed items. For example, “A and/or B” may be interpreted as “A,” “B,” or “A and B.”.

The singular forms “a,” “an,” and “the” are Intended to refer to the plural forms as well, unless the context clearly indicates otherwise. It will be further understood that the terms “comprises/comprising” and/or “includes/including” when used herein, specify the presence of stated features, integers, steps, operations, elements, and/or components, but do not preclude the presence or addition of one or more other features, integers, steps, operations, elements, components and/or groups thereof. However, the use of the term “may” herein with respect to an example or embodiment, e.g., as to what an example or embodiment may include or implement, means that at least one example or embodiment exists where such a feature is included or implemented, while all examples are not limited thereto.

Unless otherwise defined, all terms used herein including technical or scientific terms have the same meaning as commonly understood by one of ordinary skill in the art to which examples belong and based on an understanding of the disclosure of the present application. Terms, such as those defined in commonly used dictionaries, are to be interpreted as having a meaning that is consistent with their meaning in the context of the relevant art and the disclosure of the present application, and are not to be interpreted in an idealized or overly formal sense unless expressly so defined herein.

Hereinafter, examples will be described in detail with reference to the accompanying drawings. When describing the examples with reference to the accompanying drawings, like reference numerals refer to like constituent elements and a repeated description related thereto will be omitted.

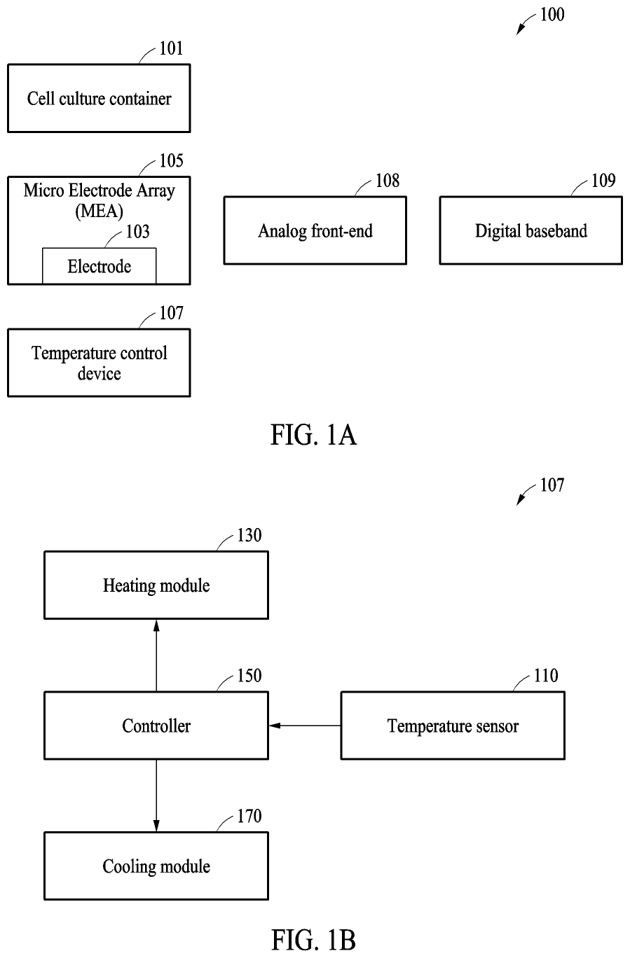

FIG. 1A illustrates an example of a micro electrode array (MEA) platform 100 and FIG. 1B illustrates a temperature control device 107 included in the MEA platform 100. Referring to FIGS. 1A and 1B, the MEA platform 100 may include a cell culture container 101, an MEA 105, and a temperature control device 107. The MEA platform 100 may further include an analog front-end 108 and a digital baseband 109.

In some examples, the cell culture container 101 may accommodate a cell culture medium for culturing biological matter, such as neurons. Hereinafter, the examples may be described with reference to neurons, however, other biological matter may be used without deviating from the spirit or scope of the examples described.

The MEA 105 may include an electrode 103 for stimulating and sensing neurons cultured in the cell culture container 101. The MEA 105 may include, for example, the electrode 103 arranged in the form of a 64×64 array but is not necessarily limited thereto. Other configurations of the electrode 103, such as, for example, 32×32 array or 16×16 array may be used without deviating from the spirit or scope of the examples described herein.

The temperature control device 107 may control the temperature of neurons by transferring heat generated by a heating source through a heating wire. In some examples, the heat may be transferred based on whether a temperature that is detected in a cell culture medium in the cell culture container 101 and/or the electrode 103 of the MEA 105 conforms to or is equal to a reference temperature for the neurons. In some examples, the temperature control device 107 may transfer heat generated by the heating source of a heating module 130 of FIG. 1B to the cell culture container 101 and/or the electrode 103 of the MEA 105, thus controlling the temperature of the neurons. The electrode 103 may include a plurality of electrodes in an array form. An example configuration of the temperature control device 107 is described with reference to FIG. 1B.

The analog front-end 108 may be configured for stimulating neurons and recording the excitation of the neurons arising from the stimulation in the form of an electrical signal. In some examples, the analog front-end 108 may include, for example, a stimulator for stimulating neurons, a signal amplifier for amplifying a signal detected in the neurons, an Analog to Digital Converter (ADC) for converting an amplified (analog) signal into a digital signal, and the like. Other configurations of the analog front-end 108 may be used without deviating from the spirit or scope of the illustrated examples described. In some examples, the analog front-end 108 may also be referred to as the input/output interface configured to provide instruction for stimulating the neurons and providing a medium for recording and displaying the results of the excitation of the neurons.

In some examples, the digital baseband 109 may perform signal processing on electrical signals of neurons that are converted into digital signals by the ADC.

Referring to FIG. 1B, the temperature control device 107 may include a temperature sensor 110, a heating module 130, and a controller 150. The temperature control device 107 may further include a cooling module 170.

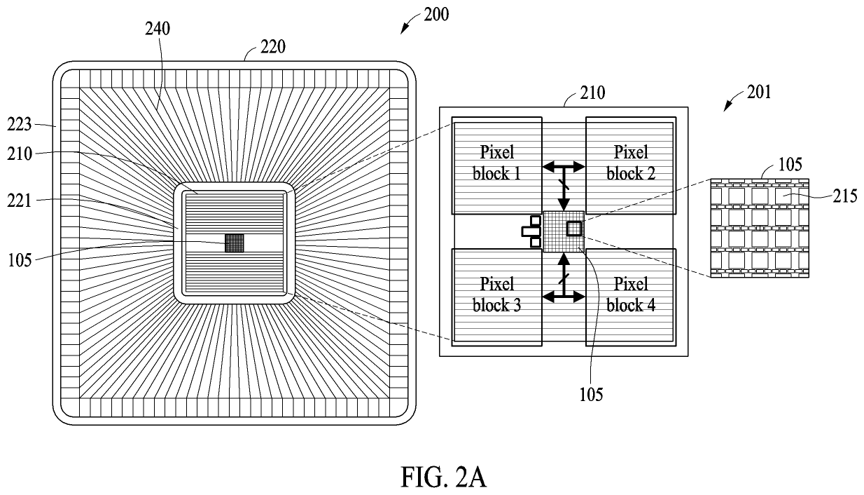

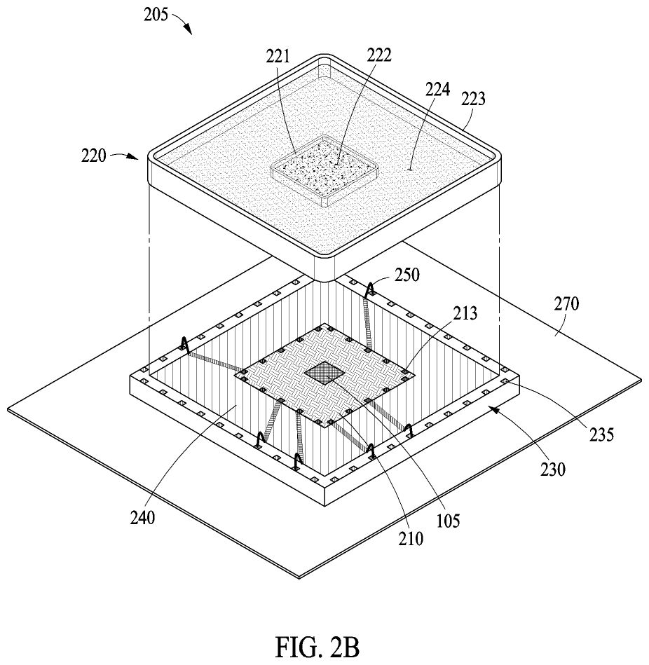

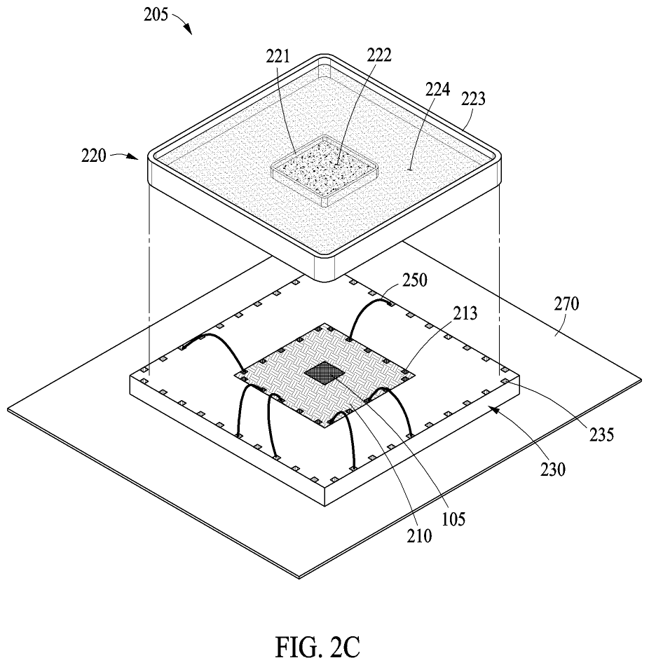

The temperature sensor 110 may detect at least one temperature of a cell culture medium for culturing neurons and the electrode 103 (e.g., an electrode 215 of FIG. 2A) of the MEA 105 for stimulating neurons. In some examples, one or more sensors may make up the temperature sensor 110. In some examples, the temperature sensor 110 may be on a chip 210, which is described hereinafter. In some examples, the temperature sensor 110 may be directly below the cell culture container 101. In some examples, the temperature sensor 110 may be below, to the left of, or the right of the electrode 103. In some examples, he temperature sensor 110 may be disposed at a location where the temperature of the neurons or the temperature of the cell culture medium may be accurately identified.

The neurons may generate an electrical signal, an optical signal, and/or a chemical signal through nerve stimulation by the electrode 103 of the MEA 105. The neurons may be cultured in the cell culture container 101 (e.g., a cell culture container 220 of FIGS. 2A-2C).

The temperature sensor 110 may detect a temperature change of at least some of the neurons and/or a temperature change of the electrode(s) 103. In some examples, the one or more temperature sensors 110 together with the controller 150 may be disposed on, for example, the bottom surface of the cell culture container 101, which accommodates the cell culture medium. In some examples, the one or more temperature sensors 110 together with the controller 150 may be disposed on, for example, the MEA 105, which may be disposed on the bottom surface of the cell culture container 101, which accommodates the cell culture medium.

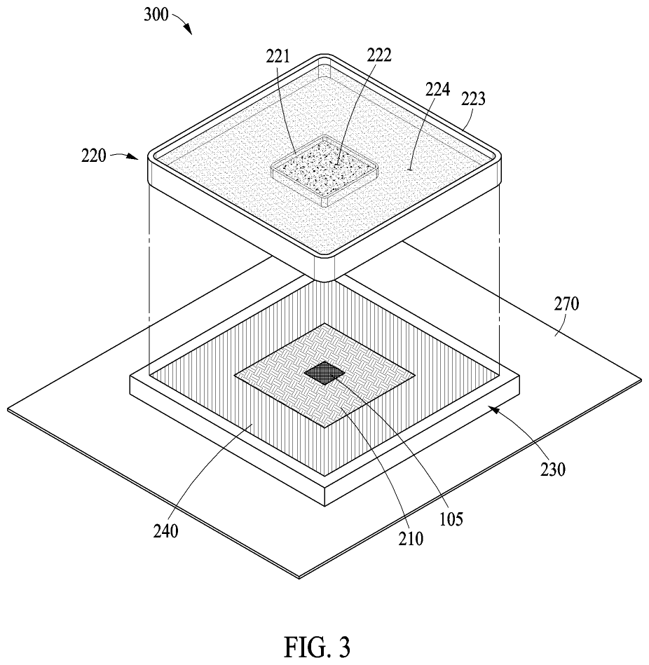

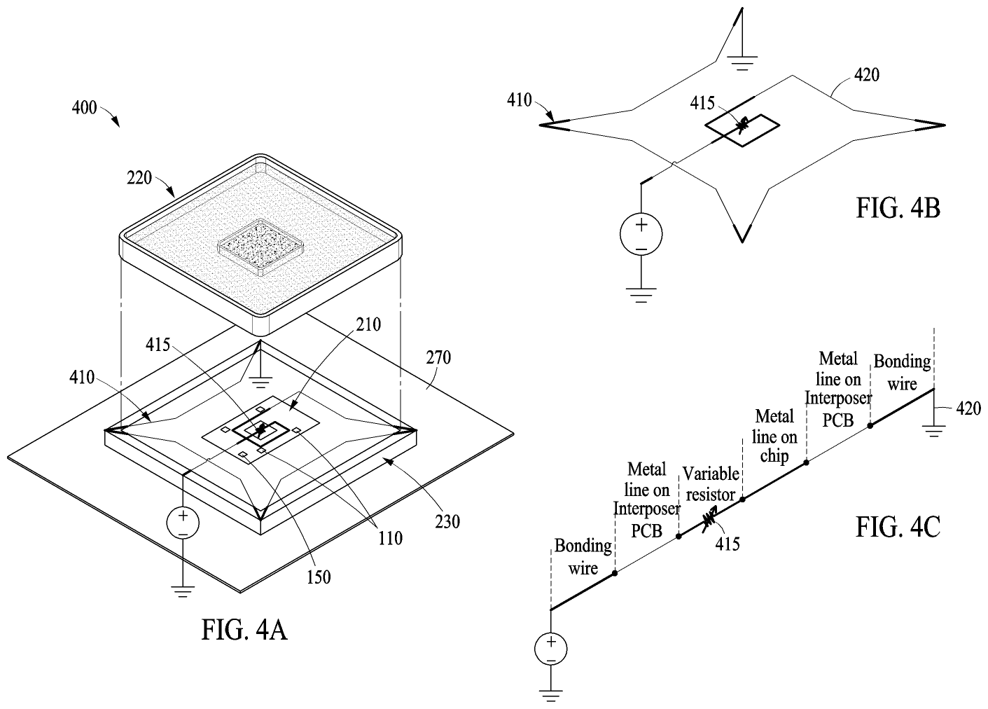

The heating module 130 may heat the electrode 103 of the MEA 105 and the cell culture medium included in the cell culture container 101, with heat generated by a heating source. The heating source may include, for example, a variable resistor (e.g., a variable resistor 415 of FIG. 4), but is not necessarily limited thereto. In some examples, the variable resistor may be configured in an on-chip form. The heating module 130 may transfer the heat generated from the heating source to the electrode 103 and/or the cell culture container 101 through a heating wire having a good characteristic of heat transfer in order to increase heating efficiency. The heating wire may be seamlessly connected as one line through materials different from one another, for example, an interposer (e.g., an interposer 240 of FIGS. 2A and 2B), a bonding wire (e.g., a bonding wire 250 of FIG. 2C), a printed circuit board (PCB) (e.g., a PCB 270 of FIG. 3), and the like. The interposer 240 may also be referred to as an ‘interposer PCB’ in that the interposer 240 is between the PCB 270 and a chip (e.g., a chip 210 of FIG. 2C). Hereinafter, ‘interposer’ and ‘interposer PCB’ may be used interchangeably and have the same meaning.

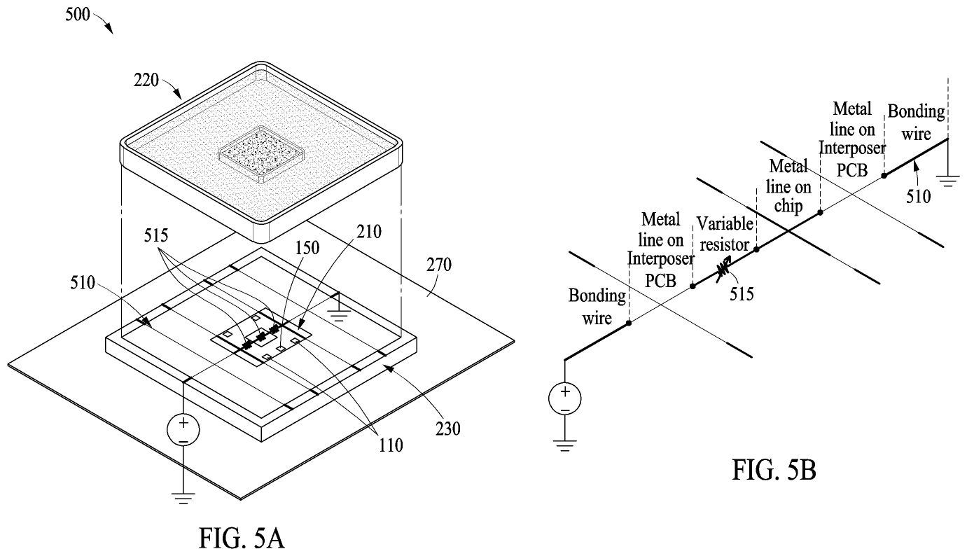

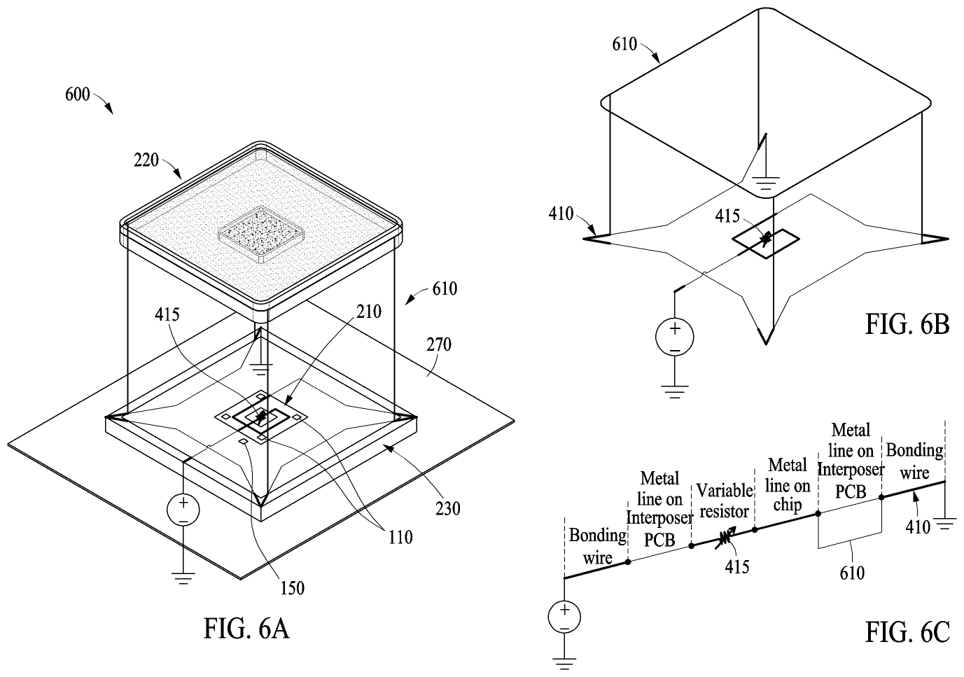

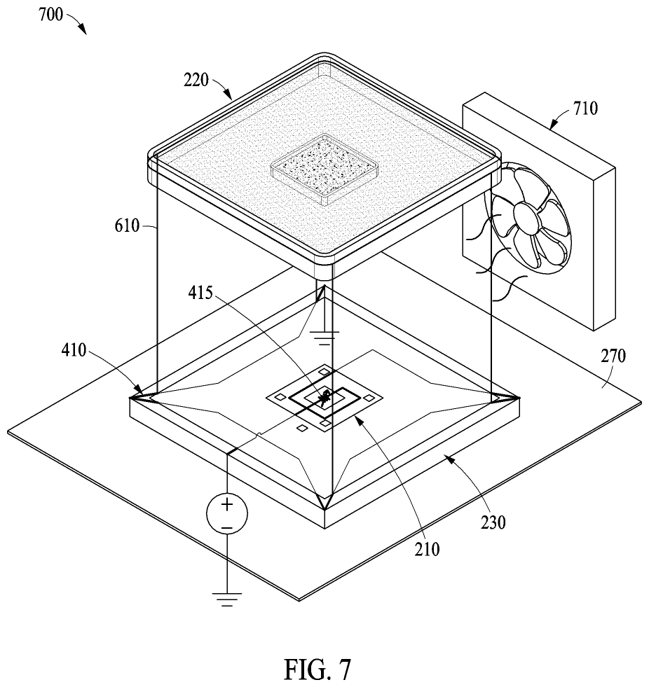

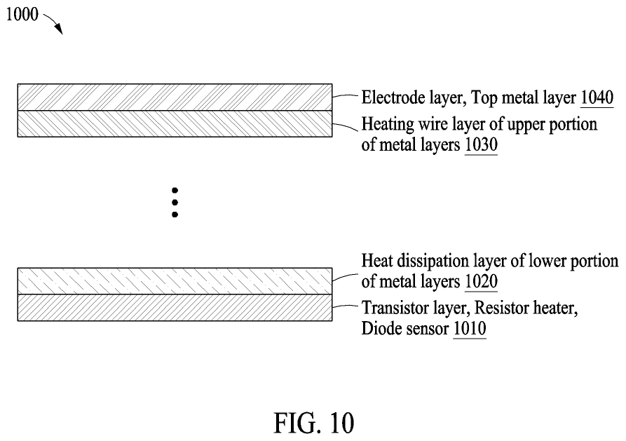

A heating wire (e.g., a metal heating wire 420 of FIG. 4, heating transfer lines 510 of FIG. 5, and/or an expanded metal heating wire 610 of FIG. 6) may be arranged to surround the electrode 103 and/or the cell culture container 101. The heating wire may be, for example, a metal heating wire having a good thermal conductivity, such as gold (Au), silver (Ag), copper (Cu), or aluminum (Al), but is not limited thereto. For example, other metals or metallic compounds may be used without deviating from the spirit or scope of the illustrated examples described. The heating wire connected to a heating source may be disposed on an upper portion of a metal layer (e.g., a heating wire layer 1030 of FIG. 10), which is at a position as close to the electrodes 103 of the MEA 105 as possible. The temperature sensor 110 and the heating module 130 may be on a chip 210 as further described with reference to FIG. 2A-2C, below, for example.

Based on whether a temperature detected by the temperature sensor 110 conforms to or is equal to a reference temperature for neurons, the controller 150 may drive the heating module 130 to transfer heat to at least one of the electrode 103 and the cell culture container 101 through the heating wire, so that the temperature of the neurons may be controlled.

The cooling module 170 may cool the electrode 103 and/or the cell culture medium in the cell culture container 101. The cooling module 170 may include, for example, at least one of a cooling fan and a heat dissipation plate. In some examples, the cooling module 170 may be omitted.

When a temperature detected by one or more temperature sensors 110 exceeds the reference temperature for neurons, the controller 150 may drive the cooling module 170 to cool the temperature of the electrode 103 and the cell culture medium to conform to the reference temperature. In addition, when a temperature detected by the one or more temperature sensors 110 is less than the reference temperature, the controller 150 may drive the heating module 130 to heat the electrode 103 and the cell culture medium to conform to the reference temperature. In some examples, the controller 150 may be configured to selectively drive one of the heating module 130 and the cooling module 170 based on a comparison of the temperature with a reference temperature for the neurons.

The neuron reference temperature may be, for example, a temperature corresponding to a human body temperature, such as 36.5 degrees to 37 degrees. In another example, when neurons to be cultured are neurons of an animal (e.g., a pig or a cow) other than a human, the reference temperature may correspond to the average body temperature (e.g., 39.2 degrees or 38.6 degrees) of the animal.

The controller 150 may determine a difference between the temperature detected by the one or more temperature sensors 110 and the reference temperature and set the resistance value of a variable resistor corresponding to a heating source according to a heating intensity that may be determined to be appropriate for the difference. In some examples, the variable resistor may be, for example, concentrated under an electrode or widely distributed in a region adjacent to a cell culture container including the electrode and a cell culture medium, but is not limited thereto.

For example, the controller 150 may drive the cooling module 170 when the temperature sensor 110 detects a temperature equal to or greater than a reference temperature and may drive the heating module 130 when a temperature equal to or less than a reference temperature is detected. Although a description in detail is as follows, a path of the heating module 130 and a path of the cooling module 170 may be configured to connect different materials in a package (e.g., a chip 210 packaged in a package 230 of FIG. 3) to a surrounding PCB (e.g., a PCB 270 of FIG. 3).

The MEA platform 100 may observe the excitation of neurons and record the excitation of the neurons in the form of an electrical signal. In addition, the MEA platform 100 may continuously monitor a cell state in real time during a cell culture process and may detect any changes in the cell state.

FIG. 2A to 2C illustrate examples of a chip in which an MEA platform 100 is packaged and FIG. 3 illustrates an example of a chip in which an MEA 105 is packaged. In some examples, as illustrated in FIGS. 2B-3, a chip 210 in which an MEA 105 is packaged may be separated from a cell culture container 220 (e.g., the cell culture container 101 of FIG. 1A) for ease of understanding. In some examples, the chip 210 in which an MEA 105 is packaged may not be separated from a cell culture container 220 (e.g., the cell culture container 101 of FIG. 1A).

Referring to FIG. 2A, illustrated is a top view 200 showing an example of the cell culture container 220 and the chip 210 in which an MEA platform 100 is packaged in a package 230. Further details regarding the package 230 are illustrated in FIGS. 2B and 2C. FIG. 2B and FIG. 2C illustrate an example of a side view 205 of the cell culture container 220 and the chip 210 in which the MEA platform 100 is packaged in the package 230.

A diagram 201 shown on the right side of the top view 200 of FIG. 2A is an enlarged view of an example of the chip 210 without packaging. Since the MEA platform 100 mounts the cell culture container 220 on the upper surface of a package 230 (illustrated in FIGS. 2B-2C), the size of the package 230 may increase. As such, when the size of the package 230 increases, the length of a bonding wire (e.g., a bonding wire 250 of FIG. 2B) may increase. An increase in the length of the bonding wire 250 may cause a decrease in yield when the package 230 is manufactured.

In an example, an interposer PCB 240 (illustrated in FIG. 2B) may be inserted between a chip bonding pad of the chip 210 and a package pin of the package 230 to reduce the length of the bonding wire connecting the pin of the package 230.

An example where the interposer PCB 240 is used for packaging is described with reference to FIG. 2B and an example where the interpose 240 is not be used for packaging is described with reference to FIG. 2C.

The chip 210 may include the MEA 105. In addition to the MEA 105, the chip 210 may include one or more temperature sensors (e.g., the temperature sensor 110 of FIG. 1B), a heating module (e.g., the heating module 130 of FIG. 1B), and components of the MEA platform 100, such as pixel blocks.

The cell culture container 220 may include a cell culture medium for culturing neurons and may be disposed on an upper portion of the chip 210.

As illustrated in FIGS. 2B-2C, the cell culture container 220 may include an inner ring 221 having a space 222, in which biological materials, such as neurons are disposed and cultured, and an outer ring 223 having a space 224 for storing a cell culture medium (e.g., a saline solution). The neurons may be, for example, brain nerve neurons or may correspond to various other nerve neurons of humans and animals.

In some examples, the interposer PCB 240 may correspond to a microcircuit board inserted between a bonding pad (e.g., the bonding pad 213 of FIG. 2B) of the chip 210 and a package pin (e.g., the package pin 235 of FIG. 2B) of the package 230. The interposer PCB 240 may supplement stability of a bonding wire (e.g., the bonding wire 250).

The interposer PCB 240 may support a stable connection between the chip 210, which is an integrated circuit (IC), and the package pin 235 of the package 230. One or more bonding wires 250 disposed on the interposer 240 may connect the chip 210 to the package pin 235 of the package 230.

The MEA platform 100 may detect the temperature of an electrode and/or the temperature of a cell culture medium by one or more temperature sensors (e.g., the temperature sensor 110 of FIG. 1B) that may be placed under the cell culture container 220. The MEA platform 100 may drive a heating module (e.g., the heating module 130 of FIG. 1B) and/or a cooling module (e.g., the cooling module 170 of FIG. 1B) through a controller (e.g., the controller 150 of FIG. 1B) according to the detected temperature. Thus, the temperature of the cell culture medium and the temperature of the electrode included in the cell culture container 220 may be controlled so that the neurons (e.g., brain neurons) may actively generate signals.

In an example, a heating wire (e.g., a metal heating wire 420 of FIG. 4, a heating transfer lines 510 of FIG. 5, and/or an expanded metal heating wire 610 of FIG. 6) that transfers heat generated by a heating source, a heat dissipation plate that transfers cool air by the cooling module 170, and/or a cooling wire may be disposed in the chip 210 and the structure of the package 230, so that the MEA platform 100 may efficiently adjust the temperature.

The heating module 130 may, for example, use heating wires disposed in various forms to transfer heat generated by a heating source. The arrangement of a heating source and the heating wire of the heating module 130 is described further with reference to FIGS. 4 to 6.

FIG. 2B illustrates an example in which the MEA platform 100 is packaged in a chip 210 using the interposer PCB 240.

In some examples of a neuron recording platform in which the cell culture container 220 is mounted on the electrode 215, similar to the MEA platform 100, the size of the package 230 may be greater than that of the chip 210. When the size of the package 230 is greater than that of the chip 210, the length of the bonding wires 250 may be increased, so that the bonding wires 250 may overlap each other. As such, when the bonding pad 213 of the chip 210 is connected to the package pin 235 only by the bonding wires 250, yield may decrease due to one or more bonding wires 250 overlapping each other. In some examples, a signal connection line between the package 230 and the chip 210 may be connected by using the interposer PCB 240 in order to prevent the decrease in the yield during packaging. In some examples, the interposer PCB 240 may be used when the difference in the size of the chip 210 and the package 230 is greater than a threshold.

As shown in FIG. 2B, in the case of a package structure with the interposer PCB 240, a heating wire, a heat dissipation plate, and/or a cooling wire may be disposed using the PCB 270 on which the chip 210, the interposer PCB 240, the bonding wire 250, and the package 230 are mounted.

FIG. 2C, illustrates an example of a diagram in which the MEA platform 100 is packaged in a chip 210 without using the interposer PCB 240.

For example, when the size of the chip 210 is similar to that of the package 230, the interposer PCB 240 may not be needed since the length of the bonding wire 250 is not so long. In some examples, the interposer PCB 240 may not be used when the difference in the size of the chip 210 and the package 230 is less than a threshold.

As shown in FIG. 2C, in the case of a package structure without the interposer PCB 240, a heating wire, a heat dissipation plate, and/or a cooling wire may be disposed using the PCB 270 on which the chip 210, the bonding wire 250, and the package 230 are mounted.

FIGS. 4A-4C illustrate examples of a heating wire arrangement structure of a heating module in an MEA platform 100. FIG. 4A illustrates an example of a diagram 400 showing a heating loop structure 410 of a heating module (e.g., the heating module 130 of FIG. 1B) of a temperature control device (e.g., the temperature control device 107 of FIG. 1A).

In some examples, as illustrated in FIGS. 4B-4C, the heating loop structure 410 may transfer heat generated by a heating source between different materials by a metal heating wire 420. Here, the different materials may be, for example, a PCB (e.g., the PCB 270 of FIG. 2B), a bonding wire (e.g., the bonding wire 250 of FIG. 2B or FIG. 2C), an interposer (e.g., the interposer 240 of FIG. 2B), and a chip (e.g., the chip 210 of FIGS. 2A-2C), but are not limited thereto. As illustrated in FIGS. 4A-4C, the heating source may be, for example, a variable resistor 415 configured in an on-chip form but is not necessarily limited thereto.

The variable resistor 415 may be, for example, concentrated under an electrode (e.g., the electrode 215 of FIG. 2A) of the MEA 105, or widely distributed in an area adjacent to the electrode 215 and the cell culture container 220. The variable resistor 415 may be formed with one variable resistor or a plurality of variable resistors.

As shown in diagrams FIGS. 4B and 4C, the heating loop structure 410 may be seamlessly connected as one by the metal heating wire 420 crossing boundaries of at least one of the interposer 240, the PCB 270, and a bonding wire (e.g., the bonding wire 250 of FIG. 2B or FIG. 2C) on the upper portion of a metal layer adjacent to the electrode 215. In some examples, the heating loop structure 410 may surround, for example, the lower surface (the bottom surface) of the cell culture container 220 including the cell culture medium but is not limited thereto.

The heating module 130 may have the heating loop structure 410 configured to surround the bottom surface of the cell culture container 220 in order to efficiently transfer the heat generated through the variable resistor 415 to the surroundings of the cell culture container 220. As shown in FIG. 4C, the metal heating wire 420, which crosses the boundaries of different materials to connect the materials, may be configured to overlap at least one of the PCB 270, the bonding wire (e.g., the bonding wire of FIG. 2B or FIG. 2C), the interposer 240, and the chip 210.

The controller 150 may determine a difference between a temperature detected by the one or more temperature sensors 110 and a reference temperature and set the resistance value of the variable resistor 415 according to a heating intensity that is determined in accordance with the difference. In this case, the one or more temperature sensors 110 may be disposed under an area corresponding to an inner ring (e.g., the inner ring 221 of FIGS. 2A-2C) of the cell culture container 220 to detect the temperature of the cell culture medium and/or the temperature of the electrode.

FIGS. 5A-5B illustrate examples of a heating wire arrangement structure of a heating module in an MEA platform 100. FIG. 5A shows an example of a heating wire arrangement structure of a heating module (e.g., the heating module 130 of FIG. 1B).

The heating module 130 may transfer heat generated by a heating source, such as the variable resistor 515, between different materials through heating transfer lines 510. Here, the different materials may be, for example, a PCB (e.g., the PCB 270 of FIG. 2B), a bonding wire (e.g., the bonding wire 250 of FIG. 2C), an interposer (e.g., the interposer 240 of FIG. 2B), and a chip (e.g., the chip 210 of FIGS. 2A-2C), but is not necessarily limited thereto.

In some examples, as illustrated in FIGS. 5A-5B, a grid of heating transfer lines 510 may be disposed on the chip and wires of the heating transfer lines 510 may intersect each other perpendicularly. In some examples, each of the heating transfer lines 510 may traverse the boundaries of the interposer 240, the PCB 270, and the bonding wire (the bonding wire 250 of FIG. 2B or FIG. 2C) 310 on the upper portion of a metal layer adjacent to an electrode (e.g., the electrode 215 of FIG. 2A). For example, each of the heating transfer lines 510 may be disposed to divide the area of the chip 210 at regular intervals, in a form perpendicular to a straight line connecting to a heating source, such as the variable resistor 515, as shown in a FIGS. 5a and 5B. The heating transfer lines 510 may be arranged at regular intervals on the bottom surface of the cell culture container 220 in order to efficiently transfer the heat generated through the variable resistor 515 to the area surrounding the cell culture container 220.

As described above with reference to FIGS. 4A-4C, the heating transfer lines 510 may be metal heating wires that cross and connect various types of materials, such as, for example, the PCB 270, the bonding wire (e.g., the bonding wire of FIG. 2B or FIG. 2C), the interposer 240, and the chip 210.

FIGS. 6A-6C illustrates an example of a heating wire arrangement structure of a heating module in an MEA platform 100. Referring to FIG. 6A, illustrated is a diagram 600 showing an example of a heating wire arrangement structure of a heating module (e.g., the heating module 130 of FIG. 1B).

As described above with reference to FIGS. 4A-4C, the heating loop structure 410 may transfer, though the metal heating wire 420, heat generated by a heating source, such as the variable resistor 415 configured in an on-chip form, between different materials. Here, the different materials may be, for example, a PCB (e.g., the PCB board 270 of FIG. 2B), a bonding wire (e.g., the bonding wire 250 of FIG. 2B or FIG. 2C), an interposer (e.g., the interposer 240 of FIG. 2B), and a chip (e.g., the chip 210 of FIG. 2B), but are not necessarily limited thereto.

The heating module 130 may include, for example, an expanded metal heating wire 610 extending from the heating loop structure 410 and expanding along the outer line of the cell culture container 220 that includes a cell culture medium, as shown in FIG. 6B.

In some examples, the expanded metal heating wire 610 may transfer heat generated through the variable resistor 415 around a circumferential portion of the cell culture container 220. Depending on the size of the cell culture container 220, the expanded metal heating wire 610 may be configured, for example, in a form of expanding from the metal line of the interposer 240 and surrounding the outer line of the cell culture container 220, as shown in FIG. 6C or in a form of expanding from the metal line of the chip 210 or the bonding wire 250 and surrounding the outer line of the cell culture container 220. As such, the expansion point of the expanded metal heating wire 610 may vary depending on the size of the cell culture container 220.

The heating module 130 may efficiently transfer, to the cell culture medium and/or the electrode, the heat generated through the variable resistor by the expanded metal heating wire 610 that surrounds the cell culture container 220.

FIG. 7 illustrates an example of an operation of a heating module and a cooling module in an MEA platform 100 and an arrangement relationship therebetween. Referring to FIG. 7, illustrated is a diagram 700 showing an example of a structure of an MEA platform 100 including both of a heating module (e.g., the heating module 130 of FIG. 1B) and a cooling module (e.g., the cooling module 170 of FIG. 1B).

A temperature control device (e.g., the temperature control device 107 of FIG. 1A) of the MEA platform 100 may include both the heating module 130 and the cooling module 170 and selectively control cooling and heating as needed.

The temperature control device may include the heating module 130 and the cooling module 170. The heating module 130 may include the expanded metal heating wire 610 in a form of expanding from the heating loop structure 410 as described in FIG. 6. The expanded metal heating wire 610 may surround the cell culture container 220 including the cell culture medium along the periphery of the cell culture container 220. The cooling module 170 may include a cooling fan 710 that is disposed outside the cell culture container 220.

For example, when the temperature of the cell culture medium in the cell culture container 220 exceeds a reference temperature, a controller (e.g., the controller 150 of FIG. 1B) of the temperature control device may drive the cooling fan 710 to reduce the temperature of the cell culture medium.

In another example, when the temperature of the cell culture medium in the cell culture container 220 is less than the reference temperature, the controller 150 may generate heat through the variable resistor 415 to increase the temperature of the cell culture medium. In this case, the heat generated through the variable resistor 415 may be quickly transferred to the cell culture container 220 through the heating loop structure 410 and the expanded metal heating wire 610 connecting to the heating loop structure 410.

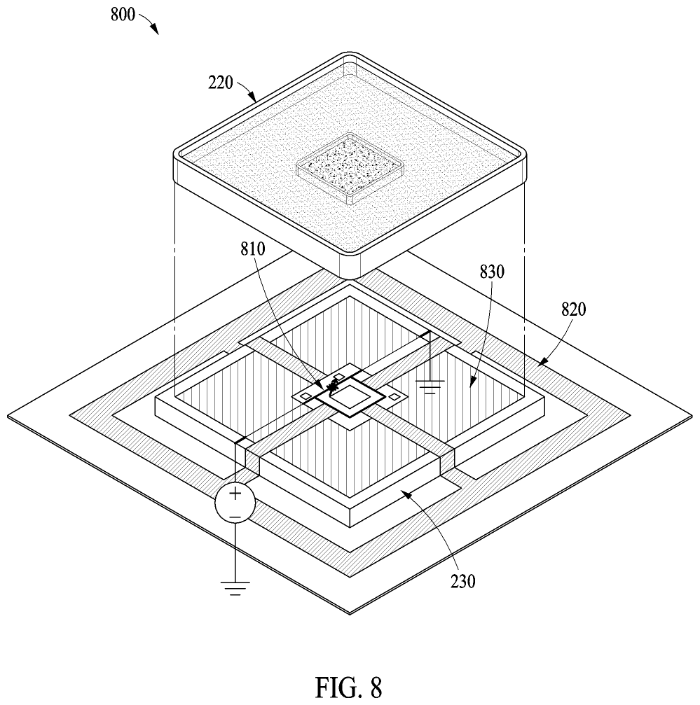

FIG. 8 illustrates an example of an MEA platform (e.g., the MEA platform 100 of FIG. 1A) including a heat dissipation plate. Referring to FIG. 8, illustrated is a diagram 800 showing an example of a heat dissipation plate 820 to cool the surroundings of a high current line 810 of the MEA platform 100.

In the MEA platform 100, in order to minimize the influence of heat in a circuit, a routing of the high current line 810, in which heat is generated more than a threshold, may be at a distance from the electrode and the cell culture medium. In some examples, the high current line 810, in which much heat is generated, may correspond to a signal line and may be identified in advance in a design stage. For example, a heat dissipation plate, such as the PCB heat dissipation plate 820, may be adjacent to the high current line 810 to prevent an increase in the temperature of an electrode due to heat generation. The heat dissipation plate 820 may correspond to a metal surface that is connected to the high current line 810 for heat dissipation. In some examples, the heat dissipation plate 820 may be disposed adjacent to a heat dissipation layer 830 in the lower portion of a metal layer of a PCB (e.g., the PCB 270 of FIG. 2B) in which the MEA platform is packaged. The heat dissipation plate may connect to at least one of a chip (e.g., the chip 210 of FIGS. 2A-2C) in which an MEA platform 100 is packaged in the package 230, an interposer (e.g., the interposer 240 of FIG. 2B), a bonding wire (e.g., the bonding wire 250 of FIG. 2B or FIG. 2C), or a PCB (e.g., the PCB 270 of FIG. 2B).

A metal surface for heat dissipation in the MEA platform may be a metal layer in a region of the PCB 270 on which a chip package is mounted. In some examples, the PCB heat dissipation plate 820 implemented on the PCB 270 may surround a chip package on a flat surface. In some examples, the PCB heat dissipation plate 820 may be disposed on the PCB 270 along an outer circumference of the package 230. In some examples, the PCB heat dissipation plate 820 may be disposed on the PCB 270 at a distance from the outer circumference of the package 230.

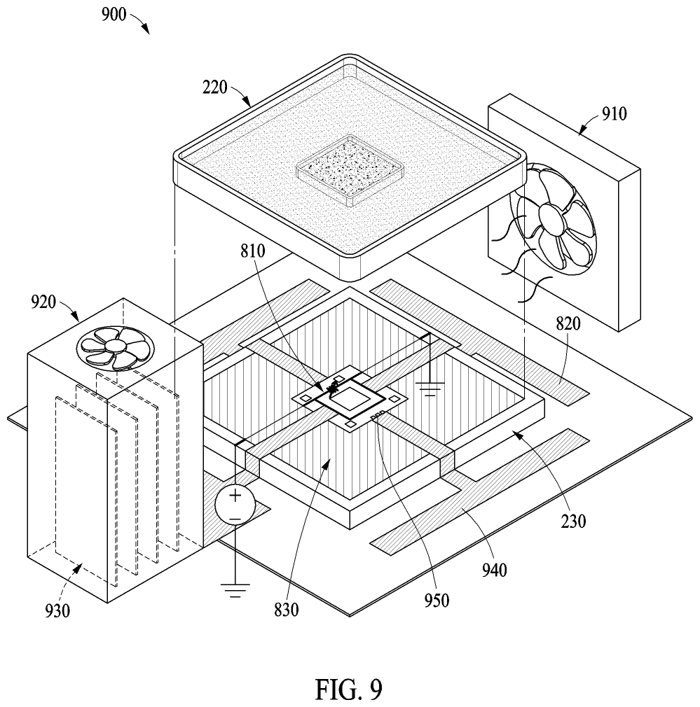

FIG. 9 illustrates an example of an MEA platform (e.g., the MEA platform 100 of FIG. 1) including a heat dissipation plate and a cooling fan. Referring to FIG. 9, illustrated is a diagram 900 showing an example of an MEA platform including cooling fans 910 and 920 and heat dissipation plates 930 and 940.

Even when a high current line 810 is equipped with a heat dissipation plate including a metal surface, such as the PCB heat dissipation plate 820, a temperature control device (e.g., the temperature control device 107 of FIG. 1A) of the MEA platform may additionally drive the cooling fans 910 and 920 of a cooling module (e.g., the cooling module 170 of FIG. 1B) according to a temperature detected by one or more temperature sensors (e.g., the temperature sensor 110 of FIG. 1B). The temperature control device may operate the cooling fans 910 and 920 in addition to the heat dissipation plates 930 and 940 to quickly cool the temperature of an electrode (e.g., the electrode 215 of FIG. 2A) of an MEA (e.g., the MEA 105 of FIG. 1A) and/or the temperature of a culture medium.

In some examples, a heat dissipation plate may correspond to a metal surface that is connected to the high current line 810 for heat dissipation. The heat dissipation plate may include, for example, the vertical heat dissipation plate 930 surrounding a chip package and/or the horizontal heat dissipation 940 disposed on a flat surface. In other examples, the heat dissipation plate may be disposed in a specific area proximal to the high current line 810.

In some examples, the heat dissipation plate may be adjacent to a heat dissipation layer 830 on a surface of a metal layer of a PCB (e.g., the PCB 270 of FIG. 2B) on which the MEA platform is packaged. The heat dissipation plate may include, for example, a heat dissipation metal plate and the heat dissipation metal plate may connect to an open-type heat dissipation pad 950 outside a chip (e.g., the chip 210 of FIG. 2) in which the MEA platform is packaged.

FIG. 10 illustrates an example of arrangement of metal layers of a chip, in which an MEA platform is packaged, and a PCB. Referring to FIG. 10, illustrated is a diagram 1000 showing metal layers between a chip (e.g., the chip 210 of FIG. 2B) and a PCB (e.g., the PCB 270 of FIG. 2B) according to an example.

A heat dissipation layer 1020 of a lower portion of metal layers may be adjacent to a PCB 1010 (e.g., the PCB 270 of FIG. 2B) on which an MEA platform including, for example, a transistor layer, a resistor heater, and a diode sensor (temperature sensor) is packaged. In some examples, the heat dissipation layer 1020 may be disposed at a distance from an electrode (e.g., the electrode 215 of FIG. 2A). In some examples, a heating wire layer 1030 of an upper portion of metal layers may be as adjacent to a top metal layer 1040 including an electrode layer.

In an example, a heat transfer efficiency may be improved by narrowing a gap between a target part to maintain a temperature (e.g., an electrode or a cell culture medium) and the heating wire layer 1030 and, at the same time, a heat dissipation effect may be improved by maintaining a wide distance between the heat dissipation layer 1020 and the top metal layer 1040 including the electrode layer.

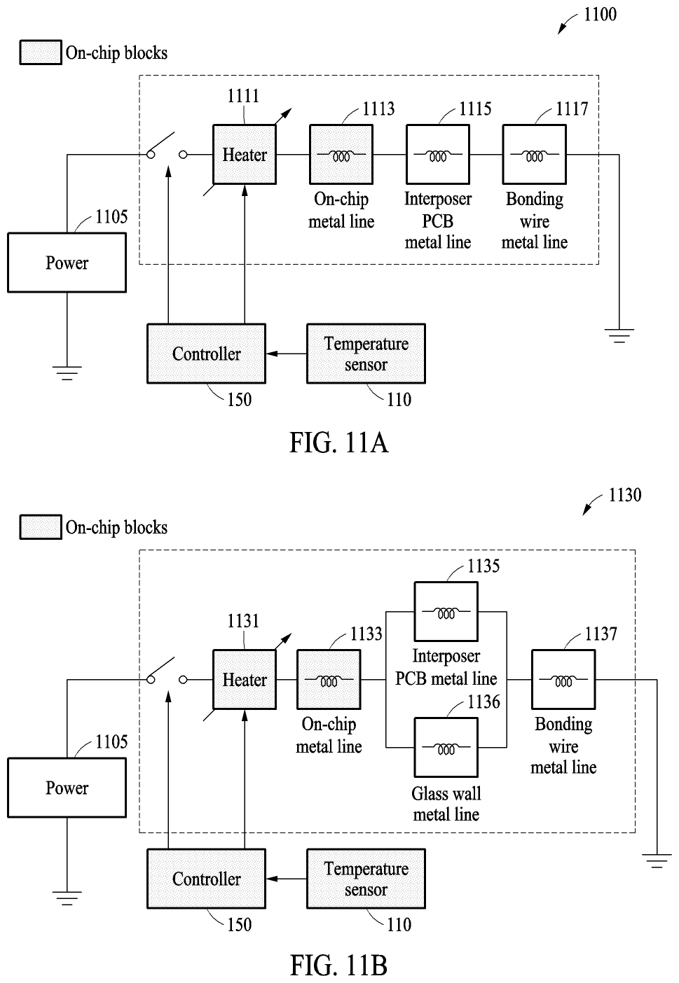

FIGS. 11A and 11B illustrate examples of a heating wire structure fora heating module in an MEA platform 100. FIG. 11A illustrates an example of a connection structure of a heat generating module 1100 that forms a closed loop to generate a heat transfer path between different materials.

Hereinafter, blocks shaded in gray in FIGS. 11A to 13B correspond to on-chip elements.

In FIG. 11A, an MEA platform may include a temperature sensor 110, a controller 150, power source 1105, and a heat generating module 1100.

When a temperature detected by the temperature sensor 110 is less than a reference temperature for neurons, the controller 150 may supply a current to a heating source 1111 by the power 1105 and transfer heat from the heating source 1111 through metal lines included in a closed loop (e.g., an on-chip metal line 1113, an interposer PCB metal line 1115, and a bonding wire metal line 1117) to heat an electrode or a cell culture medium to the reference temperature.

Different materials (e.g., the heating source 1111, the on-chip metal line 1113, the interposer PCB metal line 1115, and the bonding wire metal line 1117) in the heat generating module 1100 may be connected to one another by a metal heating wire to form a heat transfer path.

Among heat transfer paths between different materials in the heat generating module 1100, the heating source 1111 and the on-chip metal line 1113 may be implemented on a PCB, on which the MEA platform 100 is packaged, along with the temperature sensor 110 and the controller 150.

Referring to FIG. 11B, an MEA platform may include a temperature sensor 110, a controller 150, power source 1105, and a heat generating module 1130. In FIG. 11B, a heat generating module 1130 may form a heat transfer path between different materials by a closed loop that further include a glass wall metal line 1136 in addition to, for example, a heating source 1131, an on-chip metal line 1133, an interposer PCB metal line 1135, and a bonding wire metal line 1137.

When a temperature detected by the temperature sensor 110 is less than a reference temperature for neurons, the controller 150 may supply a current to the heating source 1131 by the power 1105 and transfer heat from the heating source 1131 through metal lines (e.g., the on-chip metal line 1133, the interposer PCB metal line 1135, the glass wall metal line 1136, and the bonding wire metal line 1137) included in a closed loop to heat an electrode or a cell culture medium to the reference temperature.

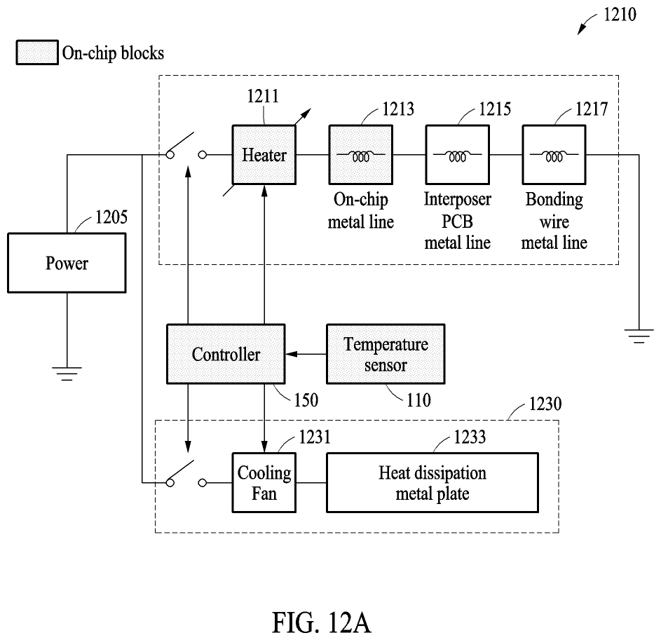

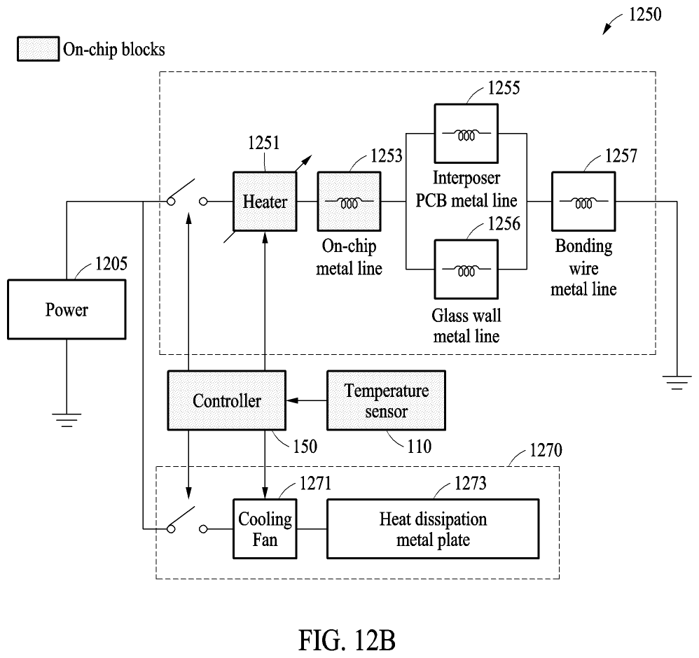

FIGS. 12A and 12B illustrate examples of a structure of a heating module and a cooling module in an MEA platform.

Referring to FIGS. 12A and 12B, a temperature control device (e.g., the temperature control device 107 of FIG. 1A) of an MEA platform according to an example may form a closed loop and include heating modules 1210 and 1250, which form a heat transfer path between different materials, and cooling modules 1230 and 1270, which include a cooling fan 1231 and a heat dissipation plate 1233. The heating module 1210 is similar to the heating module 1100 of FIG. 11A and the heating module 1250 is similar to the heating module 1130 of FIG. 11B.

When a temperature detected by a temperature sensor 110 is less than a reference temperature for neurons, a controller 150 of the temperature control device may supply a current to heating sources 1211 and 1251 by power 1205 and transfer heat generated from the heating sources 1211 and 1251 through metal lines (e.g., an on-chip metal line 1213, an interposer PCB metal line 1215, and a bonding wire metal line 1217 or an on-chip metal line 1253, an interposer PCB metal line 1255, a glass wall metal line 1256, and a bonding wire metal line 1257) included in a closed loop to heat an electrode or a cell culture medium to the reference temperature.

In another example, when a temperature detected by the temperature sensor 110 exceeds the reference temperature for neurons, the controller 150 of the temperature control device may drive the cooling modules 1230 and 1270 to cool the electrode and the cell culture medium to the reference temperature. In this case, cool air may be transferred to the electrode and/or the cell culture medium along the cooling path of cooling fans 1231 and 1271 and heat dissipation plates 1233 and 1273 to lower the temperature.

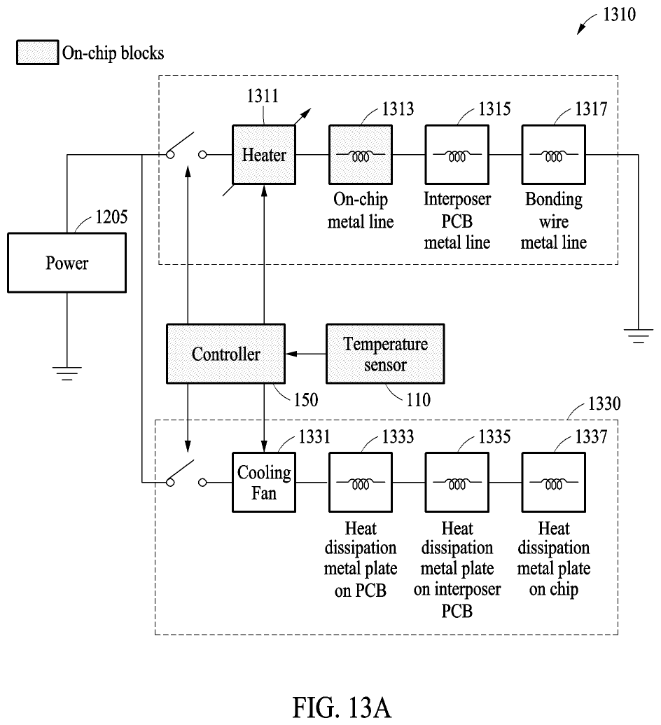

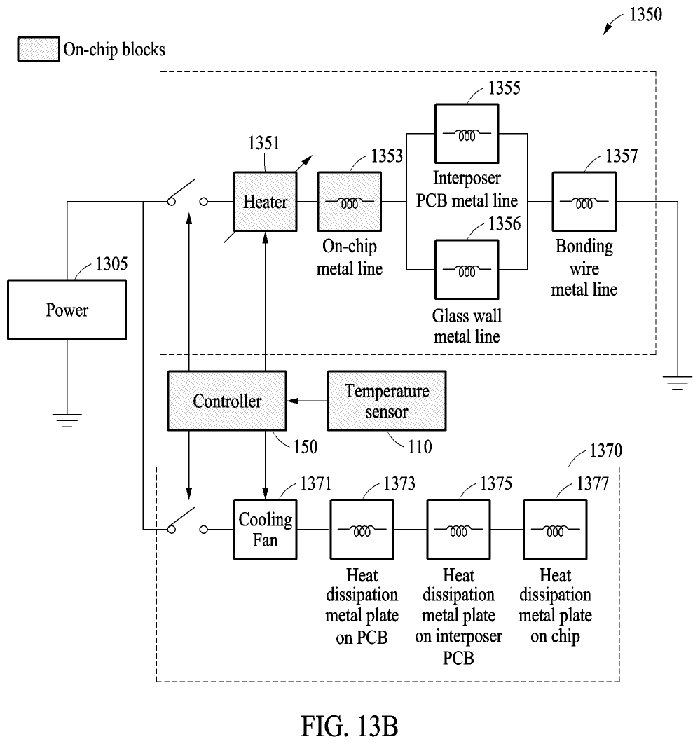

FIGS. 13A and 13B illustrate examples of structure of a heating wire of a heating module, and a cooling fan and a heat dissipation plate of a cooling module, in an MEA platform.

Referring to FIGS. 13A and 13B, a temperature control device (e.g., the temperature control device 107 of FIG. 1A) of an MEA platform according to an example may form a closed loop and include heating modules 1310 and 1350, which form a heat transfer path between different materials, and cooling modules 1330 and 1370, which include heat dissipation metal plates configured to form a cooling path between different materials.

The heating module 1310 is similar to the heating module 1100 of FIG. 11A and the heating module 1350 is similar to the heating module 1130 of FIG. 11B, the heating modules 1310 and 1350 may be referred to in the descriptions of FIGS. 11A and 11B related thereto, and are incorporated herein by reference. Thus, the above description may not be repeated here for brevity purposes.

The cooling modules 1330 and 1370 may include cooling fans 1331 and 1371, heat dissipation metal plates 1333 and 1373 on a PCB, heat dissipation metal plates 1335 and 1375 on an interposer PCB, and heat dissipation metal plates 1337 and 1377 on a chip. In this case, heat dissipation metal plates formed of different materials may be configured, for example, on a flat surface or to surround a package, similar to the heat dissipation plates 930 and 940 shown in FIG. 9.

When a temperature detected by a temperature sensor 110 exceeds a reference temperature for neurons, a controller 150 may drive the cooling modules 1330 and 1370 to cool an electrode and a cell culture medium to the reference temperature. In an example, when the cooling module 1330 is driven, cool air may be transferred along the cooling path of the cooling fan 1331 and the cooling path of the heat dissipation metal plates 1333, 1335, and 1337 to lower the temperature of the electrode and/or the cell culture medium. In another example, when the cooling module 1370 is driven, cool air may be transferred along the cooling path of the cooling fan 1371 and the cooling path of the heat dissipation metal plates 1373, 1375, and 1377 to lower the temperature of the electrode and/or the cell culture medium.

The temperature control device may be a device capable of performing bi-directional temperature control of heat generation and heat dissipation (or cooling), and include, in a chip, a heating source for heat generation (e.g., the variable resistor 415 of FIG. 4 and/or the variable resistor 515 of FIG. 5), so that a metal line inside the chip and a metal line outside and adjacent to a target part of the chip may be heated to maintain a temperature of the electrode and/or the cell culture medium. As such, the temperature control device may configure a heating conduction line as a single loop between different materials inside and outside the chip and configure the controller 150 for controlling temperature feedback as on-chip, so that heat transfer efficiency may be improved. In addition, the temperature control device may configure a cooling path to include the heat dissipation plate inside the chip and the heat dissipation plate outside the chip, similar to a heating path, and the heat dissipation metal plates 1333, 1335, and 1337 may be adjacent to a heating part where a plenty of currents flow in a package. The heat dissipation metal plates 1333, 1335, and 1337 may connect to different materials on the PCB and improve the effect on heat dissipation or cooling by driving the cooling fans 1331 and 1371 together. Here, the different materials may be, for example, a PCB (e.g., the PCB 270 of FIG. 2B), a bonding wire (e.g., the bonding wire 250 of FIG. 2B), an interposer (e.g., the interposer 240 of FIG. 2B), and a chip (e.g., the chip 210 of FIG. 2), but are not necessarily limited thereto.

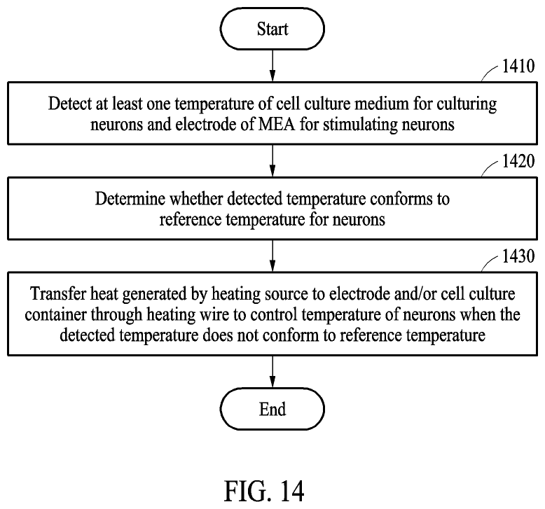

FIG. 14 illustrates an example of a method of a temperature control device. The operations of FIG. 14 may be performed in the sequence and manner as shown. However, the order of some operations may be changed, or some of the operations may be omitted, without departing from the spirit and scope of the shown example. Additionally, operations illustrated in FIG. 14 may be performed in parallel or simultaneously. One or more blocks of FIG. 14, and combinations of the blocks, can be implemented by special purpose hardware-based computer that perform the specified functions, or combinations of special purpose hardware and instructions, e.g., computer or processor instructions. For example, operations of the method may be performed by a computing apparatus (e.g., controller 150 in FIG. 1B). In addition to the description of FIG. 14 below, the descriptions of FIGS. 1-13B are also applicable to FIG. 14 and are incorporated herein by reference. Thus, the above description may not be repeated here for brevity purposes.

Referring to FIG. 14, a temperature control device (e.g., the temperature control device 107 of FIG. 1A) of an MEA platform according to an example may control the temperature of neurons through operations 1410 to 1430.

In operation 1410, the temperature control device may detect at least one temperature of a cell culture medium for culturing neurons and an electrode of an MEA for stimulating neurons.

In operation 1420, the temperature control device may determine whether a temperature detected in operation 1410 conforms to a reference temperature for neurons. The reference temperature may be, for example, human body temperature (36.5 degrees), but is not limited thereto.

In operation 1430, when the detected temperature does not conform to the reference temperature, the temperature control device may transfer heat to conform to the reference temperature. In an example, when a temperature detected in operation 1410 is lower than the reference temperature for neurons, the temperature control device may drive a heating module to heat an electrode and/or a cell culture container through a heating wire, thereby increasing the temperature of the neurons.

In another example, when a temperature detected in operation 1410 exceeds the reference temperature for neurons, the temperature control device may drive a cooling module to cool the electrode and/or the cell culture medium to the reference temperature. In another example, when a temperature detected in operation 1410 is less than the reference temperature, the temperature control device may drive a heating module including a heating source to heat the electrode and the cell culture medium to meet the reference temperature.

The heating source may include, for example, a variable resistor. The temperature control device may calculate a difference between the temperature detected in operation 1410 and the reference temperature and determine a heating intensity according to the difference. In some examples, the heating intensity corresponding to the difference between the detected temperature and the reference temperature may be stored in advance in a table. In some examples, the heating intensity may be determined in real time considering factors such as, for example, the temperature, time, and season of the surrounding environment. The MEA platform may set the resistance value of a variable resistor according to the heating intensity.

According to an example, the temperature control device may further include a cooling module for cooling at least one of the electrode and the cell culture medium. In some examples, the temperature control device may determine a difference between the detected temperature from operation 1410 and the reference temperature and determine a cooling intensity based on the difference. The cooling intensity corresponding to the difference between the detected temperature and the reference temperature may be stored in advance in a table, for example. In some examples, the cooling intensity may be determined in real time considering factors, such as, for example, the temperature, time, and season of the surrounding environment. The temperature control device may cool at least one of the electrode and the cell culture medium by setting the driving intensity of the cooling module according to a cooling intensity.

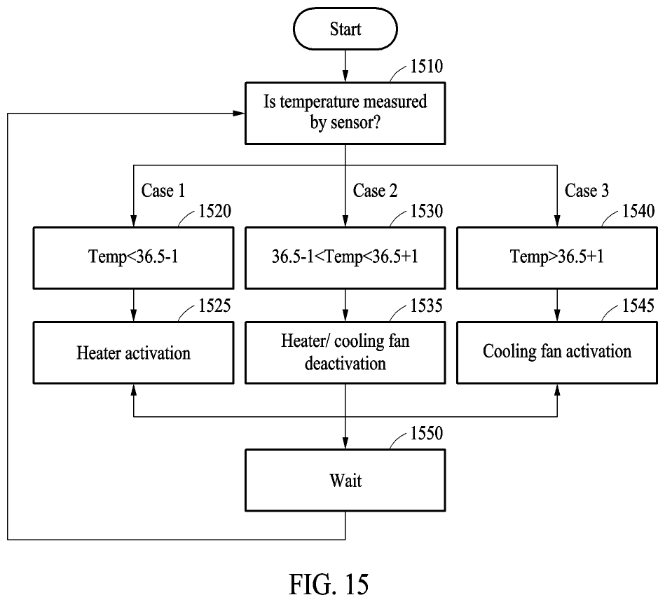

FIG. 15 illustrates an example of an operating method of a temperature control device. The operations of FIG. 15 may be performed in the sequence and manner as shown. However, the order of some operations may be changed, or some of the operations may be omitted, without departing from the spirit and scope of the shown example. Additionally, operations illustrated in FIG. 15 may be performed in parallel or simultaneously. One or more blocks of FIG. 15, and combinations of the blocks, can be implemented by special purpose hardware-based computer that perform the specified functions, or combinations of special purpose hardware and instructions, e.g., computer or processor instructions. For example, operations of the method may be performed by a computing apparatus (e.g., controller 150 in FIG. 1B). In addition to the description of FIG. 15 below, the descriptions of FIGS. 1-14 are also applicable to FIG. 15 and are incorporated herein by reference. Thus, the above description may not be repeated here for brevity purposes.

Referring to FIG. 15, in operation 1510, a temperature control device (e.g., the temperature control device 107 of FIG. 1A) of an MEA platform according to an example may measure the temperature of an electrode and the temperature of a cell culture medium by a temperature sensor (e.g., the temperature sensor 110 of FIG. 1B).

In operation 1520, the temperature control device may determine whether the temperature measured in operation 1510 is less than 36.5−1 degrees. When it is determined that the measured temperature is less than 36.5−1 degrees, in operation 1525, the temperature control device may activate or drive a heating module including a heating source. In operation 1550, the temperature control device may wait until receipt of a next measured temperature.

In operation 1510, when the temperature measured is not less than 36.5−1 degrees, the temperature control device may determine, in operation 1530, whether the measured temperature is greater than 36.5−1 degrees and less than 36.5+1 degrees. In operation 1535, when it is determined that the measured temperature is between 36.5−1 degrees and 36.5+1 degrees, the temperature control device may deactivate a heating model and a cooling module including a cooling fan. In operation 1550, the temperature control device may wait until receipt of a next measured temperature.

In operation 1540, when it is determined in operation 1530 that the measured temperature is not between 36.5−1 degrees and 36.5+1 degrees the temperature control device may determine whether the measured temperature is greater than 36.5+1 degrees. In operation 1540, when it is determined that the measured temperature is greater than 36.5+1 degrees, in operation 1545, the temperature control device may activate the cooling module including the cooling fan.

In operation 1550, the temperature control device may wait until receipt of a next measured temperature.

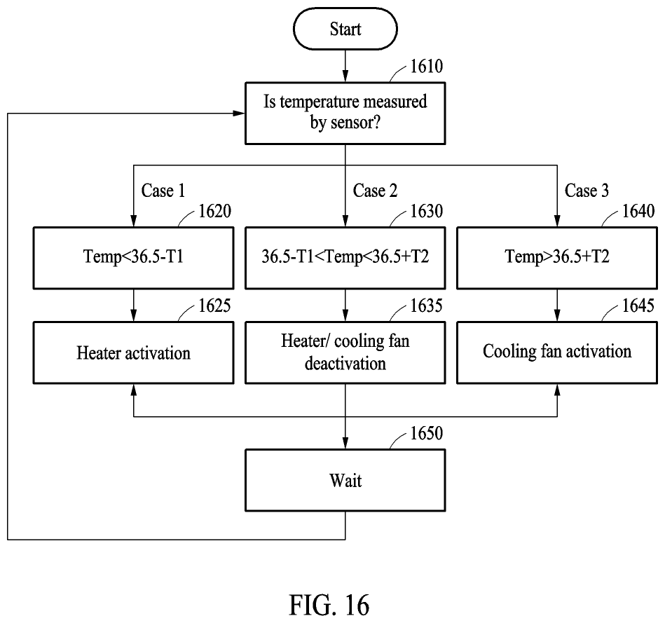

FIG. 16 illustrates an example of an operating method of a temperature control device. The operations of FIG. 16 may be performed in the sequence and manner as shown. However, the order of some operations may be changed, or some of the operations may be omitted, without departing from the spirit and scope of the shown example. Additionally, operations illustrated in FIG. 16 may be performed in parallel or simultaneously. One or more blocks of FIG. 16, and combinations of the blocks, can be implemented by special purpose hardware-based computer that perform the specified functions, or combinations of special purpose hardware and instructions, e.g., computer or processor instructions. For example, operations of the method may be performed by a computing apparatus (e.g., controller 150 in FIG. 1B). In addition to the description of FIG. 16 below, the descriptions of FIGS. 1-15 are also applicable to FIG. 16 and are incorporated herein by reference. Thus, the above description may not be repeated here for brevity purposes.

Referring to FIG. 16, in operation 1610, a temperature control device (e.g., the temperature control device 107 of FIG. 1A) of an MEA platform according to an example may measure the temperature of an electrode and the temperature of a cell culture medium by a temperature sensor.

In operation 1620, the temperature control device may determine whether the temperature measured in operation 1610 is less than 36.5−T1 degrees. In operation 1625, when it is determined that the measured temperature is less than 36.5−T1 degrees, the temperature control device may activate or drive a heating module including a heating source. In operation 1650, the temperature control device may wait until receipt of a next measured temperature.

In operation 1630, when the temperature measured in operation 1610 is not less than 36.5−T1 degrees, the temperature control device may determine whether the measured temperature is greater than 36.5−T1 degrees and less than 36.5+T2 degrees. In operation 1635, when it is determined that the measured temperature is between 36.5−T1 degrees and 36.5+T2 degrees, the temperature control device may deactivate the heating module and a cooling module including a cooling fan. Here, T1 and T2 may be adjusted to various values according to an example. In operation 1650, the temperature control device may wait until receipt of a next measured temperature.

When it is determined that the temperature measured in operation 1630 is not between 36.5−T1 degrees and 36.5+T2 degrees, then, in operation 1640, the temperature control device may determine whether the measured temperature is greater than 36.5+T2 degrees. In operation 1640, when it is determined that the measured temperature is greater than 36.5+T2 degrees, in operation 1645, the temperature control device may activate the cooling module including the cooling fan. In operation 1650, the temperature control device may wait until receipt of a next measured temperature.

The computing apparatuses, the electronic devices, the processors, the memories, and components described herein with respect to FIGS. 1-16 are implemented by or representative of hardware components. Examples of hardware components that may be used to perform the operations described in this application where appropriate include controllers, sensors, generators, drivers, memories, comparators, arithmetic logic units, adders, subtractors, multipliers, dividers, integrators, and any other electronic components configured to perform the operations described in this application. In other examples, one or more of the hardware components that perform the operations described in this application are implemented by computing hardware, for example, by one or more processors or computers. A processor or computer may be implemented by one or more processing elements, such as an array of logic gates, a controller and an arithmetic logic unit, a digital signal processor, a microcomputer, a programmable logic controller, a field-programmable gate array, a programmable logic array, a microprocessor, or any other device or combination of devices that is configured to respond to and execute instructions in a defined manner to achieve a desired result. In one example, a processor or computer includes, or is connected to, one or more memories storing instructions or software that are executed by the processor or computer. Hardware components implemented by a processor or computer may execute instructions or software, such as an operating system (OS) and one or more software applications that run on the OS, to perform the operations described in this application. The hardware components may also access, manipulate, process, create, and store data in response to execution of the instructions or software. For simplicity, the singular term “processor” or “computer” may be used in the description of the examples described in this application, but in other examples multiple processors or computers may be used, or a processor or computer may include multiple processing elements, or multiple types of processing elements, or both. For example, a single hardware component or two or more hardware components may be implemented by a single processor, or two or more processors, or a processor and a controller. One or more hardware components may be implemented by one or more processors, or a processor and a controller, and one or more other hardware components may be implemented by one or more other processors, or another processor and another controller. One or more processors, or a processor and a controller, may implement a single hardware component, or two or more hardware components. A hardware component may have any one or more of different processing configurations, examples of which include a single processor, independent processors, parallel processors, single-instruction single-data (SISD) multiprocessing, single-instruction multiple-data (SIMD) multiprocessing, multiple-instruction single-data (MISD) multiprocessing, and multiple-instruction multiple-data (MIMD) multiprocessing.

The methods illustrated in FIGS. 14-16 that perform the operations described in this application are performed by computing hardware, for example, by one or more processors or computers, implemented as described above implementing instructions or software to perform the operations described in this application that are performed by the methods. For example, a single operation or two or more operations may be performed by a single processor, or two or more processors, or a processor and a controller. One or more operations may be performed by one or more processors, or a processor and a controller, and one or more other operations may be performed by one or more other processors, or another processor and another controller. One or more processors, or a processor and a controller, may perform a single operation, or two or more operations.

Instructions or software to control computing hardware, for example, one or more processors or computers, to implement the hardware components and perform the methods as described above may be written as computer programs, code segments, instructions or any combination thereof, for individually or collectively instructing or configuring the one or more processors or computers to operate as a machine or special-purpose computer to perform the operations that are performed by the hardware components and the methods as described above. In one example, the instructions or software include machine code that is directly executed by the one or more processors or computers, such as machine code produced by a compiler. In another example, the instructions or software includes higher-level code that is executed by the one or more processors or computer using an interpreter. The instructions or software may be written using any programming language based on the block diagrams and the flow charts illustrated in the drawings and the corresponding descriptions herein, which disclose algorithms for performing the operations that are performed by the hardware components and the methods as described above.

The instructions or software to control computing hardware, for example, one or more processors or computers, to implement the hardware components and perform the methods as described above, and any associated data, data files, and data structures, may be recorded, stored, or fixed in or on one or more non-transitory computer-readable storage media. Examples of a non-transitory computer-readable storage medium include read-only memory (ROM), random-access programmable read only memory (PROM), electrically erasable programmable read-only memory (EEPROM), random-access memory (RAM), dynamic random access memory (DRAM), static random access memory (SRAM), flash memory, non-volatile memory, CD-ROMs, CD-Rs, CD+Rs, CD-RWs, CD+RWs, DVD-ROMs, DVD-Rs, DVD+Rs, DVD-RWs, DVD+RWs, DVD-RAMs, BD-ROMs, BD-Rs, BD-R LTHs, BD-Res, blue-ray or optical disk storage, hard disk drive (HDD), solid state drive (SSD), flash memory, a card type memory such as multimedia card micro or a card (for example, secure digital (SD) or extreme digital (XD)), magnetic tapes, floppy disks, magneto-optical data storage devices, optical data storage devices, hard disks, solid-state disks, and any other device that is configured to store the instructions or software and any associated data, data files, and data structures in a non-transitory manner and provide the instructions or software and any associated data, data files, and data structures to one or more processors or computers so that the one or more processors or computers can execute the instructions. In one example, the instructions or software and any associated data, data files, and data structures are distributed over network-coupled computer systems so that the instructions and software and any associated data, data files, and data structures are stored, accessed, and executed in a distributed fashion by the one or more processors or computers.

While this disclosure includes specific examples, it will be apparent after an understanding of the disclosure of this application that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner, and/or replaced or supplemented by other components or their equivalents.

What is claimed is:

1. A micro electrode array (MEA) platform comprising:
a cell culture container configured to accommodate a cell culture medium to culture neurons;
an MEA comprising an electrode configured to sense the neurons; and
a temperature control device configured to control a transfer of heat generated by a heating source through a heating wire, based on whether a temperature of the cell culture medium or the electrode matches a reference temperature for the neurons.

2. The MEA platform of claim 1, wherein the temperature control device comprises:
a temperature sensor configured to detect the temperature of the cell culture medium or the electrode;
a heating module configured to heat the electrode or the cell culture medium by the heat generated by the heating source; and
a controller configured to control the heating module based on whether the temperature is equal to the reference temperature, and to transfer the heat to at least one of the electrode or the cell culture container through the heating wire.

3. The MEA platform of claim 2, wherein
the temperature control device further comprises a cooling module configured to cool at the electrode or the cell culture medium; and
the controller is further configured to:
drive the cooling module to cool a temperature of the electrode and a temperature of the cell culture medium, in response to the temperature being greater than the reference temperature; and
drive the heating module to increase the temperature of the electrode and the temperature of the cell culture medium, in response to determining that the temperature being less than the reference temperature.

4. The MEA platform of claim 1, wherein
the MEA platform is packaged in a chip form on a printed circuit board (PCB) using one or more of an interposer and a bonding wire, and
the cell culture container is disposed on an upper surface of the chip.

5. The MEA platform of claim 4, wherein
the heating source comprises an on-chip variable resistor, and
the controller is further configured to determine a difference between the temperature and the reference temperature and to set a resistance value of the variable resistor according to the difference.

6. The MEA platform of claim 2, wherein the heating module comprises a heating loop made of a metal wire,
wherein the heating loop crosses boundaries of an interposer, a printed circuit board (PCB), or a bonding wire in an upper portion of a metal layer positioned adjacent to the electrode.

7. The MEA platform of claim 6, wherein the heating loop structure is shaped to surround a bottom surface of the cell culture container.

8. The MEA platform of claim 6, further comprising:
an expanded metal heating wire extending from the heating loop to be disposed along a perimeter of the cell culture container,
wherein the expanded metal heating wire is configured to transfer heat generated through the variable resistor to surroundings of the cell culture container.

9. The MEA platform of claim 8, further comprising a cooling module comprising a cooling fan disposed outside the cell culture container,
wherein the controller is further configured to:
drive the cooling fan to lower the temperature of the cell culture medium, in response to the temperature of the cell culture medium being greater than the reference temperature; and
generate heat through the variable resistor to increase the temperature of the cell culture medium, in response to the temperature of the cell culture medium being less than the reference temperature.

10. The MEA platform of claim 2, wherein the heating module is configured to transfer the heat generated by the heating source, by heat transfer lines, and
wherein each of the heat transfer lines is disposed to cross boundaries of at least one of an interposer, a printed circuit board (PCB), or a bonding wire in an upper portion of a metal layer positioned adjacent to the electrode.

11. The MEA platform of claim 2, wherein the temperature control device further comprises:
a heat dissipation plate disposed adjacent to a high current line where the heat is generated more than a threshold amount, and
the heat dissipation plate is disposed adjacent to a heat dissipation layer in a lower portion of a metal layer of a printed circuit board (PCB) on which the MEA platform is packaged.

12. The MEA platform of claim 11, wherein
the temperature control device further comprises a cooling fan disposed outside the cell culture container, and
the controller is further configured to cool the cell culture medium or the heat dissipation plate by activating the cooling fan.

13. The MEA platform of claim 11, wherein the heat dissipation plate:
comprises a vertical heat dissipation plate configured in a form of surrounding a chip package or a horizontal heat plate configured on a plane; and
is connected to a chip in which the MEA platform is packaged, an interposer, a bonding wire, or a PCB.

14. An method of operating a temperature control device of a micro electrode array (MEA) platform, the method comprising:
detecting a temperature of at least one of a cell culture medium culturing neurons or an electrode of an MEA configured to sense the neurons;
determining whether the detected temperature is equal to a reference temperature for the neurons; and
controlling a transfer of heat generated by a heating source through a heating wire, in response to the detected temperature not matching the reference temperature.

15. The method of claim 14, wherein the controlling of the transfer of the heat comprises:
driving a cooling module to cool the electrode and the cell culture medium, in response to the detected temperature being greater than the reference temperature; and
driving a heating module comprising the heating source to heat the electrode and the cell culture medium, in response to the detected temperature being lesser than the reference temperature.

16. The method of claim 14, wherein
the heating source comprises a variable resistor, and
the controlling of the transfer of the heat comprises:
determining a difference between the detected temperature and the reference temperature;
determining a heating intensity according to the difference; and
setting a resistance value of the variable resistor according to the heating intensity.

17. The method of claim 14, wherein the transfer of the heat comprises:
determining a difference between the detected temperature and the reference temperature;
determining a cooling intensity according to the difference; and
cooling at least one of the electrode or the cell culture medium according to the cooling intensity.

18. A device comprising:
a chip installed on a surface of a printed circuit board (PCB), the chip comprising
a micro electrode array (MEA) configured to sense signals generated by biological matter,
a temperature sensor configured to measure a temperature of the biological matter,
a heating module configured to heat the biological matter based on the temperature, and
a controller configured to control the heating module;
a cell culture container disposed on an upper surface of the chip, the cell culture container configured to store the biological matter in a cell culture medium; and
an interface configured to output the signals.

19. The device of claim 18, wherein the heating module comprises a variable resistor disposed under the MEA, and the controller is further configured to vary a resistance of the variable resistor according to a difference of the temperature and a reference temperature for the biological matter.

20. The device of claim 18, further comprising a cooling module configured to cool the biological matter based on the temperature, and the controller is further configured to selectively drive one of the heating module and the cooling module based on a comparison of the temperature with a target temperature for the biological matter.
